(19) United States
(12) Patent Application Publication
(10) Pub. No.: US 2022/0249351 A1
(43) Pub. Date: Aug. 11, 2022

(54) COSMETIC

(71) Applicant: Noevir Co., Ltd., Hyogo (JP)

(72) Inventors: Sumiko KIZAKI, Shiga (JP); Jyunya YOSHIMOTO, Shiga (JP)

(73) Assignee: Noevir Co., Ltd., Hyogo (JP)

(21) Appl. No.: 17/616,401

(22) PCT Filed: Jun. 6, 2019
(86) PCT No.: PCT/JP2019/022458

§ 371 Date: Dec. 3, 2021

(51) Int. Cl. A61K 8/73; A61K 8/02; A61K 8/27

(52) U.S. Cl. CPC A61K 8/732; A61K 8/022; A61Q 1/02; A61K 2800/654; A61K 2800/621; A61K 8/27

(57) ABSTRACT

Provided is a cosmetic containing a composite particle containing a starch alkenyl succinate metal salt coated with 0.05 to 2 parts by mass of zinc oxide to 1 part by mass of the starch alkenyl succinate metal salt. The starch alkenyl succinate metal salt may be at least one selected from the group consisting of a starch octenyl succinate metal salt, a starch decenyl succinate metal salt, a starch dodecenyl succinate metal salt, a starch tetradecenyl succinate metal salt, a starch hexadecenyl succinate metal salt, and a starch octadecenyl succinate metal salt.

### TECHNICAL FIELD

The present invention relates to a cosmetic.

### BACKGROUND

Cosmetics are applied to a person's skin for use, and thus they are required to be resistant to sweat and sebum from the skin, and they are generally required to not result in makeup being lifted away from the skin due to oil or makeup deterioration. In order to satisfy such requirements, studies are being carried out to prevent makeup deterioration by adding various improvements to powders used in cosmetics.

For example, a method may be cited in which inorganic porous powders such as porous silica and porous spherical magnesium carbonate are blended into a cosmetic, but there may be a problem in that these powders absorb moisture on the skin and cause drying and itching of the skin due to a lack of emollient components in the skin (Patent Documents 1 and 2). Furthermore, organic powders such as acrylic polymers are used as sebum adsorbents, but it is expected that development will be carried out into materials with even better sebum adsorption capability than these sebum adsorbents.

Meanwhile, in the field of cosmetics, zinc oxide is used as a white pigment and an ultraviolet shielding material due to the particle size thereof. For example, it is known that fine-particle zinc oxide reacts with fatty acids contained in sebum to solidify the sebum and suppress the spread thereof, thereby improving the long-lasting property of makeup (Patent Document 3). However, such fine-particle zinc oxide has a disadvantage in that it results in a stiff feeling when blended into a cosmetic and the touch feel thereof is poor. Therefore, if it is possible to obtain zinc oxide particles having an excellent touch feel and high sebum solidifying capability, they can be an extremely useful material in the field of cosmetics.

Furthermore, a composite powder is also known in which a (acrylate/ethylhexyl acrylate) crosspolymer is coated with 0.2 to 2 parts by mass of zinc oxide to 1 part by mass of the crosspolymer (Patent Document 4), but there is a need for powders and cosmetics that exhibit even higher sebum solidifying capability.

### PRIOR ART DOCUMENTS

### Patent Documents

Patent Document 1: Japanese Unexamined Patent Application Publication No. 2009-137806
Patent Document 2: Japanese Unexamined Patent Application Publication No. 2006-096706
Patent Document 3: Japanese Unexamined Patent Application Publication No. H8-41379
Patent Document 4: Japanese Patent No. 6467100

### SUMMARY

### Technical Problem

An object of the present invention is to provide a cosmetic containing a composite particle that exhibits high sebum solidifying capability.

### Solution to Problem

One or more aspects of the present invention are as follows.

[1] A cosmetic contains a composite particle containing a starch alkenyl succinate metal salt coated with 0.05 to 2 parts by mass of zinc oxide to 1 part by mass of the starch alkenyl succinate metal salt.

[2] The cosmetic according to [1], wherein, in the composite particle, the starch alkenyl succinate metal salt is coated with 0.1 to 1.5 parts by mass of the zinc oxide to 1 part by mass of the starch alkenyl succinate metal salt.

[3] The cosmetic according to [1] or [2], wherein the starch alkenyl succinate metal salt includes at least one selected from the group consisting of a starch octenyl succinate metal salt, a starch decenyl succinate metal salt, a starch dodecenyl succinate metal salt, a starch tetradecenyl succinate metal salt, a starch hexadecenyl succinate metal salt, and a starch octadecenyl succinate metal salt.

[4] The cosmetic according to [3], wherein the starch alkenyl succinate metal salt includes a starch octenyl succinate metal salt.

[5] The cosmetic according to any of [1] to [4], wherein the starch alkenyl succinate metal salt includes at least one selected from the group consisting of a starch alkenyl succinate sodium, a starch alkenyl succinate potassium, a starch alkenyl succinate magnesium, a starch alkenyl succinate calcium, a starch alkenyl succinate barium, a starch alkenyl succinate copper, a starch alkenyl succinate aluminum, a starch alkenyl succinate iron, and a starch alkenyl succinate zirconium.

[6] The cosmetic according to [5], wherein the starch alkenyl succinate metal salt includes starch alkenyl succinate aluminum.

[7] The cosmetic according to any of [1] to [6], wherein the starch alkenyl succinate metal salt includes starch octenyl succinate aluminum.

[8] The cosmetic according to any of [1] to [7], wherein an average particle size of the starch alkenyl succinate metal salt is 1 to 30 μm.

[9] The cosmetic according to any of [1] to [8], wherein an average particle size of the zinc oxide is 10 to 200 nm.

[10] The cosmetic according to any of [1] to [9], wherein the zinc oxide includes zinc oxide subjected to hydrophobization treatment.

### Effects of Invention

According to the present invention, it is possible to provide a cosmetic which contains a composite particle exhibiting high sebum solidifying capability, and which does not easily deteriorate due to sebum and has a considerable effect on the long-lasting property of makeup.

### DESCRIPTION OF EMBODIMENTS

Embodiments for carrying out the present invention will be described in detail below. However, the present invention is not limited to the embodiments below.

The composite particle contained in the cosmetic of the present invention contains a starch alkenyl succinate metal salt and zinc oxide that coats the starch alkenyl succinate metal salt, and it is preferable that the starch alkenyl succinate metal salt be coated with 0.05 to 2 parts by mass of zinc oxide to 1 part by mass of the starch alkenyl succinate metal salt. This composite particle preferably has a form in which the particle surface of the starch alkenyl succinate metal salt is coated with zinc oxide.

The starch alkenyl succinate metal salt in the present invention is a metal salt of a starch ester of a succinic acid having an alkenyl group. In an alkenyl succinate, the carbon number of an alkenyl group is preferably 5 to 22, more preferably 10 to 20, and even more preferably 8 to 18. Examples of an alkenyl succinate include an octenyl succinate, decenyl succinate, dodecenyl succinate, tetradecenyl succinate, hexadecenyl succinate, and octadecenyl succinate. In particular, a metal salt of a starch octenyl succinate may be preferably used. The metal salt is preferably a monovalent, divalent, or trivalent metal salt, and examples include sodium, potassium, magnesium, calcium, barium, copper, aluminum, iron, and zirconium. In particular, an aluminum salt, calcium salt, or sodium salt of a starch alkenyl succinate may be preferably used, with an aluminum salt being more preferable. From among them, starch octenyl succinate aluminum may be preferably used.

Examples of starches that can be used in the present invention include natural starches such as maize starch, tapioca starch, rice starch, or Amaranthus starch, and those obtained by fractionation of small particles of wheat starch or potato starch. Furthermore, modified starches of the aforementioned may be used, and examples include acid-degraded starches, oxidized starches, etherified, esterified, or cross-linked starch derivatives, and heat moisture treated starches.

Regarding the average particle size of the starch alkenyl succinate metal salt used for the composite particle of the present invention, in terms of compositing it is favorable for the average particle size to be preferably 1 to 30 μm and more preferably 2 to 25 μm. Here, the average particle size is based on volume according to a laser diffraction/scattering type of particle size distribution measuring device.

The shape of the starch alkenyl succinate metal salt is not particularly limited, but may be a spherical, elliptical, polygonal, or crushed-like shape, for example.

The starch alkenyl succinate metal salt can be produced according to a typical production method.

The zinc oxide used for the composite particle of the present invention is not particularly limited as long as it can be blended into a cosmetic. The shape of the zinc oxide is not particularly limited, but from the viewpoint of the sebum solidifying capability, the average particle size is preferably 10 to 200 nm, more preferably 15 to 100 nm, and even more preferably 15 to 50 nm. Here, the average particle size is obtained by measuring the particle sizes of 1000 particles according to the principles of electron microscopy and averaging by the number of particles.

For the zinc oxide, untreated zinc oxide can be used as it is but it is preferable to use zinc oxide that has been subjected to hydrophobization treatment. The hydrophobization treatment agent is not particularly limited, and examples thereof include dimethicone, methylhydrogen polysiloxane, and metallic soap. From among these hydrophobization treatment agents, it is preferable to use dimethicone, methylhydrogen polysiloxane, a copolymer thereof, or a mixture thereof. It is sufficient for the coating amount of the hydrophobization treatment agent to be an amount adequate for carrying out hydrophobization treatment on the zinc oxide. Specifically, the mass ratio of the zinc oxide and the hydrophobization treatment agent is preferably 85:15 to 99:1, and more preferably 90:10 to 98:2.

In the composite particle used in the cosmetic of the present invention, the coating amount of zinc oxide is 0.05 parts by mass or more, more preferably 0.1 parts by mass or more, and even more preferably 0.2 parts by mass or more, to 1 part by mass of the starch alkenyl succinate metal salt. The coating amount of zinc oxide is 2 parts by mass or less, and more preferably 1.5 parts by mass or less, to 1 part by mass of the starch alkenyl succinate metal salt. With each range, the sebum solidifying capability can be further increased, and deterioration due to sebum can be further prevented.

The coating amount of zinc oxide is 0.05 to 2 parts by mass, and more preferably 0.1 to 1.5 parts by mass, to 1 part by mass of the starch alkenyl succinate metal salt.

Various types of hitherto known methods can be used to coat zinc oxide onto the starch alkenyl succinate metal salt, and a physicochemical mixing/grinding method (dry or wet type) or a chemical deposition method can be selected, for example. In terms of the sebum solidifying capability of the composite particle, a dry type of mixing/grinding method may be preferably used.

For the composite particle of the present invention, it is possible to use a composite particle in which one type or two or more types selected from among the starch alkenyl succinate metal salts described above are coated with zinc oxide. Untreated zinc oxide, zinc oxide subjected to hydrophobization treatment, or a combination thereof may be used for the zinc oxide. one type of composite particle may be blended alone or a combination of two or more types may be blended into the cosmetic of the present invention, with the composite particle being a combination of one type or two or more types selected from among the starch alkenyl succinate metal salts described above and zinc oxide.

As a result of containing the aforementioned composite particle, the cosmetic of the present invention is able to have sebum solidifying capability and exhibit an excellent effect on the long-lasting property of makeup.

As the amount of the composite particle to be blended into the cosmetic, 0.5 to 90% by mass relative to the total amount of the cosmetic is preferable. When this blend amount is less than 0.5% by mass, the sebum solidifying capability of the composite particle is unlikely to be effectively exhibited in the cosmetic. Meanwhile, when the composite particle exceeding 90% by mass is blended into the cosmetic, pharmaceutical preparation tends to become difficult.

Although applications of the cosmetic of the present invention are not particularly limited, in terms of the effect thereof, the cosmetic is suitable for use in makeup cosmetics such as foundations, and base cosmetics, sunscreen cosmetics, and the like.

The dosage form of the cosmetic of the present invention is not particularly limited, and examples thereof include powder cosmetics, emulsified cosmetics, and double-layer cosmetics.

In addition to the composite particle, the cosmetic of the present invention may also have the following blended therein as appropriate: water-based components, moisturizing agents, oil-based components, colorants, surfactants, ultraviolet absorbing agents, thickening agents, beautifying components, fragrances, polymeric substances, antibacterial and antifungal agents, alcohols, powders, scrubbing agents, biologically derived components, and the like, which are normally blended into cosmetics as necessary.

### EXAMPLES

Hereinafter, the present invention will be described in specific terms using examples, but the scope of the present invention is not limited thereby. Note that the blend amounts are “% by mass” unless specifically stated otherwise.

### (Composite Particle)

The core particles and zinc oxide listed in Table 1 were mixed for two hours using a blender to thereby obtain composite particles. Observation of the composite particles using a microscope found that the core particles were coated with zinc oxide. Note that zinc oxide having an average particle size of 25 nm and treated with 5% dimethicone was used.

In Table 1, the coating amount of zinc oxide is given as a mass percentage of the total amount of core particles and zinc oxide.

The average particle size of the core particles prior to being coated with zinc oxide is shown in Table 1.

### (Oleic Acid Solidification Test)

(a) Amounts of 5 g of oleic acid (PM810: Miyoshi Oil & Fat Co., Ltd.) and 1 g of ion-exchanged water are measured in a 20-mL beaker and heated to 35 to 40° C. using a hot plate set to 40° C.

(b) An amount of 0.5 g of composite particles is added to (a) and stirred for 30 seconds using a spatula until homogeneous.

(c) After stirring, a stirring element is quickly introduced and stirring is carried out using a stirrer (200 rpm). The time from the start of stirring to the gelation of the oleic acid progressing and the rotation of the stirring element stopping was measured and used as the solidification time.

TABLE 1

| | | | Average | | | |
|---|---|---|---|---|---|---|
| | | | particle | | Zinc oxide | Solidification |
| | | | size | | coating | time |
| | | Core particle | (μm) | Shape | amount (%) | (seconds) |
| Composite | 1 | Starch octenyl succinate aluminum | 5-20 | Polygon | 20 | 0 |
| particle | 2 | (Octie: Nippon Starch Chemical Co., Ltd.) | | | 30 | 0 |
| | 3 | | | | 50 | 0 |
| | 4 | | | | 0 | Did not solidify |
| | 5 | Starch octenyl succinate calcium | 14 | Polygon | 50 | 0 |
| | 6 | (Experimental product) | | | 0 | Did not solidify |
| | 7 | (Acrylate/ethylhexyl acrylate) crosspolymer | 8 | Porous | 30 | 12 |
| | 8 | (Techpolymer ACP-8C: Sekisui Kasei Co., Ltd.) | | sphere | 50 | 4 |
| | 9 | Acrylate copolymer | 8 | Core | 30 | 93 |
| | 10 | (GANZPEARL GBM-55COS: Aica Kogyo Co., Ltd.) | | shell | 50 | 30 |
| | 11 | Acrylate crosspolymer | 8 | Porous | 30 | 142 |
| | 12 | (GANZPEARL GMP-0820: Aica Kogyo Co., Ltd.) | | sphere | 50 | 66 |
| | 13 | Methyl methacrylate crosspolymer | 8 | Porous | 30 | 72 |
| | 14 | (GANZPEARL GMP-0800: Aica Kogyo Co., Ltd.) | | sphere | 50 | 57 |
| | 15 | Methyl methacrylate crosspolymer | 15 | Hollow | 30 | 80 |
| | 16 | (Matsumoto Microsphere MHB-R: Matsumoto Yushi-Seiyaku Co., Ltd) | | | 50 | 68 |
| | 17 | Oil-encapsulated methyl methacrylate crosspolymer | 13 | Elliptical | 30 | 62 |
| | 18 | (Matsumoto Microsphere M-311: Matsumoto Yushi-Seiyaku Co., Ltd) | | bowl | 50 | 47 |
| | 19 | Nylon-6 | 13 | Porous | 30 | 90 |
| | 20 | (Nylon powder TR-1: Toray Industries, Inc.) | | sphere | 50 | 68 |
| | 21 | (Dimethicone/vinyl dimethicone) crosspolymer | 7 | Perfect | 30 | 86 |
| | 22 | (GANZPEARL SIG-070: Aica Kogyo Co., Ltd.) | | sphere | 50 | 55 |
| | 23 | Urethane resin | 15 | Perfect | 30 | 102 |
| | 24 | (GRANPEARL GU-1500: Aica Kogyo Co., Ltd.) | | sphere | 50 | 78 |
| | 25 | (Styrene/DVB) copolymer | 6 | Perfect | 30 | 50 |
| | 26 | (GANZPEARL GS-0605: Aica Kogyo Co., Ltd.) | | sphere | 50 | 22 |

As shown in Table 1, it is indicated that the composite particles 1 to 3 of starch octenyl succinate aluminum and zinc oxide have an extremely quick oleic acid solidification time and exhibit high sebum solidifying capability. Furthermore, it is indicated that the composite particles 5 of starch octenyl succinate calcium and zinc oxide also have an extremely short oleic acid solidification time and exhibit high sebum solidifying capability.

Next, using the composite particles 1 shown in Table 1, a press-type foundation was prepared by a usual method using the formulation shown in Table 2.

### (Evaluation Method)

Three evaluators with expertise in makeup cosmetics used Example 1 and Comparative Example 1 each on half of a face, evaluated deterioration due to sebum after three hours in a room having high temperature and high humidity conditions, and evaluated by consensus which was superior according to the criteria below.

### [Deterioration Due to Sebum]

∘: Unlikely to deteriorate due to sebum

x: Likely to deteriorate due to sebum

TABLE 2

| | | Comparative |
|---|---|---|
| Component | Example 1 | Example 1 |
| Dimethicone-treated fine- | 8 | 8 |
| particle titanium oxide | | |
| Pigment-grade titanium oxide | 10 | 10 |
| Dimethicone-treated talc | Amount required for | |
| | total amount to be 100 | |
| Dimethicone-treated mica | 10 | 10 |
| Fluoride-treated mica | 7 | 7 |
| Fine-particle zinc oxide | 15 | 15.6 |
| (average particle size 25 nm) | | |
| Nylon powder | 5 | 5 |
| Poly (methyl methacrylate) | 5 | 5 |
| Composite particle 1 | 3 | — |
| Starch octenyl succinate aluminum | — | 2.4 |
| Dimethicone-treated yellow iron oxide | 2 | 2 |
| Dimethicone-treated red iron oxide | 0.6 | 0.6 |
| Dimethicone-treated black iron oxide | 0.4 | 0.4 |
| Dimethicone | 5 | 5 |
| Cross-linked silicone polymer | 1.5 | 1.5 |
| Cetyl ethylhexanoate | 3 | 3 |
| Ethylhexyl methoxycinnamate | 4 | 4 |
| Phenoxyethanol | 0.5 | 0.5 |
| Tocopherol | 0.1 | 0.1 |
| Deterioration due to sebum | ◯ | X |

As shown in Table 2, it is indicated that Example 1 having composite particles blended therein is unlikely to deteriorate due to sebum.

Next, using the composite particles 1 to 3 shown in Table 1, various types of cosmetics were prepared according to the procedures below.

### [Example 2] Powder Foundation

TABLE 3

| | Component | Blend amount (mass %) |
|---|---|---|
| 1 | Composite particle | 5.0 |
| 2 | Dimethicone-treated titanium oxide | 5.0 |
| 3 | Dimethicone treated fine- | 10.0 |
| | particle titanium oxide | |
| 4 | Magnesium stearate | 5.0 |
| 5 | Dimethicone treated talc | Amount required for |
| | | total amount to be 100 |
| 6 | Dimethicone-treated yellow iron oxide | Appropriate amount |
| 7 | Dimethicone-treated red iron oxide | Appropriate amount |
| 8 | Dimethicone-treated black iron oxide | Appropriate amount |
| 9 | Poly (alkyl acrylate) | 5.0 |
| 10 | Dimethicone-treated mica | 15.0 |
| 11 | Dimethicone | 10.0 |
| 12 | Paraffin wax | 1.0 |
| 13 | 2-ethylhexyl stearate | 5.0 |

Production method: Components 1 to 10 are mixed and homogenized, then components 11 to 13 which are heated and dissolved to 75° C. are added, and the mixture is kneaded. The mixture is pulverized using an atomizer, sieved, and then pressed into a metal dish.

### [Example 3] Oil-Based Foundation (Compact Type)

TABLE 4

| | Component | Blend amount (mass %) |
|---|---|---|
| 1 | Composite particle | 5.0 |
| 2 | Dimethicone treated talc | Amount required for |
| | | total amount to be 100 |
| 3 | Dimethicone treated mica | 15.0 |
| 4 | Dimethicone-treated yellow iron oxide | Appropriate amount |
| 5 | Dimethicone-treated red iron oxide | Appropriate amount |
| 6 | Dimethicone-treated black iron oxide | Appropriate amount |
| 7 | Solid paraffin | 3.0 |
| 8 | Microcrystalline wax | 6.0 |
| 9 | Beeswax | 2.0 |
| 10 | Vaseline | 12.0 |
| 11 | Squalane | 6.0 |

Production method: Components 1 to 6 are mixed and homogenized, then components 7 to 11 which are heated and dissolved are added, and the mixture is kneaded by a roll mill. The kneaded mixture is re-melted, stirred slowly to raise foam, and then cooled and poured into a container at 60° C. to be allowed to cool and solidify.

### [Example 4] Blemish Concealer Stick

TABLE 5

| | Component | Blend amount (mass %) |
|---|---|---|
| 1 | Talc | 5.8 |
| 2 | Kaolin | Amount required for |
| | | total amount to be 100 |
| 3 | Mica | 3.0 |
| 4 | Yellow iron oxide | Appropriate amount |
| 5 | Red iron oxide | Appropriate amount |
| 6 | Black iron oxide | Appropriate amount |
| 7 | Composite particle 2 | 4.0 |
| 8 | Titanium dioxide | 6.0 |
| 9 | Solid paraffin | 3.0 |
| 10 | Microcrystalline wax | 7.0 |
| 11 | Vaseline | 15.0 |
| 12 | Methylphenyl polysiloxane | 3.0 |
| 13 | Squalane | 5.0 |
| 14 | Isopropyl palmitate | 17.0 |

Production method: Components 9 to 14 are dissolved at 85° C., and components 1 to 8 which are mixed and homogenized are added while stirring. The mixture is ground and dispersed using a colloid mill, and after degassing the mixture is poured into a container at 70° C. and cooled.

### [Example 5] O/W Emulsified Foundation

TABLE 6

| | Component | Blend amount (mass %) |
|---|---|---|
| 1 | Talc | 3.0 |
| 2 | Yellow iron oxide | Appropriate amount |
| 3 | Red iron oxide | Appropriate amount |
| 4 | Black iron oxide | Appropriate amount |
| 5 | Composite particle 3 | 3.0 |
| 6 | Kaolin | 0.5 |
| 7 | Polyoxyethylene sorbitan monostearate | 0.9 |
| 8 | Triethanolamine | 1.0 |
| 9 | Propylene glycol | 10.0 |
| 10 | Purified water | Amount required for |
| | | total amount to be 100 |
| 11 | Stearic acid | 2.2 |
| 12 | Isohexadecyl alcohol | 7.0 |
| 13 | Glyceryl monostearate | 2.0 |
| 14 | Liquid lanolin | 2.0 |
| 15 | Liquid paraffin | 8.0 |

Production method: Component 6 is dispersed in component 9 and added to component 10, the mixture is processed using a homomixer at 70° C., then components 7 and 8 are added and the mixture is stirred sufficiently. Components 1 to 5 which are mixed and homogenized are added to the mixture, which is then mixed using a homomixer until homogeneous. Components 11 to 15 which are heated and dissolved at 75° C. and homogenized are added, and the mixture is emulsified using a homomixer and then cooled.

### [Example 6] W/O Emulsified Foundation

TABLE 7

| | Component | Blend amount (mass %) |
|---|---|---|
| 1 | Sericite | 10.0 |
| 2 | Yellow iron oxide | Appropriate amount |
| 3 | Red iron oxide | Appropriate amount |
| 4 | Black iron oxide | Appropriate amount |
| 5 | Composite particle | 2.0 |
| 6 | Decamethyl cyclopentasiloxane | 12.0 |
| 7 | Polyoxyethylene-modified | 4.0 |
| | dimethylpolysiloxane | |
| 8 | Purified water | Amount required for |
| | | total amount to be 100 |
| 9 | 1,3-butylene glycol | 5.0 |
| 10 | Sodium chloride | 2.0 |

Production method: Components 8 to 10 are heated, dissolved, and homogenized, components 1 to 5 are added, and the mixture is homogeneously dispersed using a homomixer. Components 6 and 7 which are homogeneously dissolved are added, and the mixture is emulsified using a homomixer and then cooled.

### [Example 7] O/W Emulsified Sunscreen Cream

TABLE 8

| | Component | Blend amount (mass %) |
|---|---|---|
| 1 | Oxybenzone | 2.0 |
| 2 | Octyl paramethoxycinnamate | 5.0 |
| 3 | Squalane | 10.0 |
| 4 | Vaseline | 5.0 |
| 5 | Stearyl alcohol | 3.0 |
| 6 | Stearic acid | 3.0 |
| 7 | Glycerin monostearate ester | 3.0 |
| 8 | Ethyl polyacrylate | 1.0 |
| 9 | Purified water | Amount required for |
| | | total amount to be 100 |
| 10 | 1,3-butylene glycol | 7.0 |
| 11 | Fine-particle titanium oxide | 3.0 |
| 12 | Composite particle 3 | 2.0 |
| 13 | Triethanolamine | 1.0 |

Production method: Components 9 to 13 are mixed and then homogeneously dispersed using a homomixer, after which components 1 to 8 which are heated and dissolved to 75° C. are added, and the mixture is emulsified using a homomixer and then cooled.

### [Example 8] W/O Emulsified Sunscreen Emulsion

TABLE 9

| | Component | Blend amount (mass %) |
|---|---|---|
| 1 | Octyl paramethoxycinnamate | 5.0 |
| 2 | Oxybenzone | 3.0 |
| 3 | 4-tert-butyl-4′-methoxybenzoylmethane | 1.0 |
| 4 | Dimethicone-treated fine-particle | 5.0 |
| | titanium oxide | |
| 5 | Composite particle 2 | 5.0 |
| 6 | Squalane | 20.0 |
| 7 | Dimethicone | Amount required for |
| | | total amount to be 100 |
| 8 | Silicone resin | 2.0 |
| 9 | Glycerin diisostearate | 2.0 |
| 10 | Organic modified montmorillonite | 0.5 |
| 11 | Purified water | 28.5 |
| 12 | 1,3-butylene glycol | 5.0 |

Production method: Components 1 to 10 are heated, dissolved, and mixed and then homogeneously dispersed using a homomixer, components 11 and 12 which are homogeneously dissolved are added, and the mixture is emulsified using a homomixer and then cooled.

1. A cosmetic comprising: a composite particle containing a starch alkenyl succinate metal salt coated with 0.05 to 2 parts by mass of zinc oxide to 1 part by mass of the starch alkenyl succinate metal salt.

2. The cosmetic according to claim 1, wherein, in the composite particle, the starch alkenyl succinate metal salt is coated with 0.1 to 1.5 parts by mass of the zinc oxide to 1 part by mass of the starch alkenyl succinate metal salt.

3. The cosmetic according to claim 1, wherein the starch alkenyl succinate metal salt includes at least one selected from the group consisting of a starch octenyl succinate metal salt, a starch decenyl succinate metal salt, a starch dodecenyl succinate metal salt, a starch tetradecenyl succinate metal salt, a starch hexadecenyl succinate metal salt, and a starch octadecenyl succinate metal salt.

4. The cosmetic according to claim 3, wherein the starch alkenyl succinate metal salt includes a starch octenyl succinate metal salt.

5. The cosmetic according to claim 1, wherein the starch alkenyl succinate metal salt includes at least one selected from the group consisting of a starch alkenyl succinate sodium, a starch alkenyl succinate potassium, a starch alkenyl succinate magnesium, a starch alkenyl succinate calcium, a starch alkenyl succinate barium, a starch alkenyl succinate copper, a starch alkenyl succinate aluminum, a starch alkenyl succinate iron, and a starch alkenyl succinate zirconium.

6. The cosmetic according to claim 5, wherein the starch alkenyl succinate metal salt includes a starch alkenyl succinate aluminum.

7. The cosmetic according to claim 1, wherein the starch alkenyl succinate metal salt includes starch octenyl succinate aluminum.

8. The cosmetic according to claim 1, wherein an average particle size of the starch alkenyl succinate metal salt is 1 to 30 μm.

9. The cosmetic according to claim 1, wherein an average particle size of the zinc oxide is 10 to 200 nm.

10. The cosmetic according to claim 1, wherein the zinc oxide includes zinc oxide subjected to hydrophobization treatment.

11. The cosmetic according to claim 1, wherein an average particle size of the starch alkenyl succinate metal salt is 1 to 30 and an average particle size of the zinc oxide is 10 to 200 nm.

12. The cosmetic according to claim 1, wherein an average particle size of the starch alkenyl succinate metal salt is 1 to 30 μm, an average particle size of the zinc oxide is 10 to 200 nm, and the zinc oxide includes zinc oxide subjected to hydrophobization treatment.

13. The cosmetic according to claim 2, wherein an average particle size of the starch alkenyl succinate metal salt is 1 to 30 an average particle size of the zinc oxide is 10 to 200 nm, and the zinc oxide includes zinc oxide subjected to hydrophobization treatment.

14. The cosmetic according to claim 13, wherein the starch alkenyl succinate metal salt includes starch octenyl succinate aluminum.
